(19) United States
(12) Patent Application Publication
(10) Pub. No.: US 2009/0191616 A1
(43) Pub. Date: Jul. 30, 2009

(54) Biosensor structure and fabricating method thereof

(75) Inventors: Yi-Chun Lu, Taipei County (TW); Tri-Rung Yew, Hsinchu City (TW); Hwan-You Chang, Hsinchu City (TW); Ya-Shuan Chuang, Taipei City (TW)

Correspondence Address: J C PATENTS, INC., 4 VENTURE, SUITE 250, IRVINE, CA, 92618, US

(73) Assignee: National Tsing Hua University, Hsinchu City (TW)

(21) Appl. No.: 12/231,071

(22) Filed: Aug. 27, 2008

(30) Foreign Application Priority Data
Jan. 30, 2008 (TW) 97103637

(51) Int. Cl. C12M 1/00

(52) U.S. Cl. 435/287.2; 427/2.11

(57) ABSTRACT

A biosensor structure and a method for fabricating the same are described. The biosensor structure for detecting at least a single cell includes a substrate with an insulating surface, a conductive layer and a plurality of capture molecules. The conductive layer is disposed on the substrate, and has a first pattern and a second pattern separated from each other. The first pattern includes a plurality of first finger configurations, and the second pattern includes a plurality of second finger configurations, so as to form interdigitated array. The capture molecules are immobilized on the conductive layer, such that the cell that is bound specifically to the capture molecules on two adjacent first and second finger configurations is detected. The biosensor structure is feasible for real-time (<3 min), specific, and quantitative targeted cell detection down to a single cell.

### CROSS-REFERENCE TO RELATED APPLICATION

This application claims the priority benefit of Taiwan application serial no. 97103637, filed Jan. 30, 2008. The entirety of the above-mentioned patent application is hereby incorporated by reference herein and made a part of this specification.

### BACKGROUND OF THE INVENTION

1. Field of the Invention

The present invention generally relates to a biosensor structure and a fabricating method thereof, and in particular, to a biosensor structure applied to various types of organism with specific antigen, especially bacteria cells, and a fabricating method thereof.

2. Description of Related Art

Rapid and sensitive detection of pathogenic bacteria is a key requirement for efficient and effective prevention and identification of problems related to health and safety. Although this concept is simple, this goal still encounters major challenges. Established methods for pathogen detection include polymerase chain reaction (PCR), culture and colony counting methods, and immunology-based methods. However, these methods still face the issues such as extensive analysis time and process complexity.

An alternative method for bacteria detection is the use of biosensors, which combines a biological recognition mechanism with a physical transduction technique. The biosensors are classified into bioaffinity sensors and biocatalytic sensors based on the type of the biological recognition element to be determined. The transduction of biosensor may be micromechanical, electrochemical, piezoelectric, thermometric, magnetic, or optical. Among these approaches, the electrochemical transduction methods, such as amperometry, impedimetry, potentiometry, are much less time-consuming and more sensitive than other techniques. Various approaches to detect bacteria on the basis of electromechanical systems have been presented.

Radke and Alocilja (Radke, S. M., Alocilja, E. C., 2005. Biosens. Bioelectron. 20, 1662-1667 and Radke, S. M., Alocilja, E.C., 2005. IEEE Sens. J. 5 (4), 744-750) disclosed that the sensitivities of using impedimetry approach could be down to 10⁴-10⁷ CFU/ml in pure culture within 5 min based on the detection of Escherichia coli (E. coli O157:H7) by measuring the bacteria impedance at different frequency (100 Hz to 10 MHz) with bacteria immobilized on SiO₂.

Muhammad-Tahir and Alocilja (Muhammad-Tahir, Z., Alocilja, E. C., 2003. Biosens. Bioelectron. 18, 813-819 and Muhammad-Tahir, Z., Alocilja, E. C., 2004. Biosyst. Eng. 88 (2) 145-151) have demonstrated another approach to measure the resistance drop due to the electron transfer facilitated by the polyaniline-labeled antibody between electrodes. Results show that this approach exhibits the sensitivity of being able to detect about 81 CFU/ml in 6 min and 79 CFU/ml in 10 min, respectively.

As mentioned above, these approaches cannot preserve higher sensitivity and diminish detection time simultaneously. Although previous attempts have been made to address the detection limit and analysis time, such efforts have not been sufficient to adequately fulfill the increasing requirements for a real-time and highly-sensitive detection.

### SUMMARY OF THE INVENTION

Accordingly, the present invention is directed to a biosensor structure for a real-time, specific and quantitative detection down to a single cell.

The present invention is also directed to a method for fabricating a biosensor structure of this invention.

The biosensor structure of this invention for detecting at least a single cell includes a substrate with an insulating surface, a conductive layer and a plurality of capture molecules. The conductive layer is disposed on the substrate, and has a first pattern and a second pattern separated from each other. The first pattern includes a plurality of first finger configurations, and the second pattern includes a plurality of second finger configurations, so as to form interdigitated array. The capture molecules are immobilized on the conductive layer, such that the cell which is bound specifically to the capture molecules on two adjacent first and second finger configurations is detected.

According to an embodiment of the present invention, the capture molecules are antibodies or antibody fragments, which bind to a specific antigen presented by the cell. A self-assembled monolayer is further disposed between the conductive layer and the capture molecules, wherein the self-assembled monolayer includes 11-mercaptoundecanoic acid. The conductive layer may comprise gold (Au), aluminium (Al) or platinum (Pt). In addition, an adhesion layer can be disposed between the substrate and the conductive layer.

According to an embodiment of the present invention, the substrate comprises a silicon layer and a dielectric layer. The dielectric layer disposed on the silicon layer has a thickness of 5-500 nm, of which the material can be silicon dioxide, silicon nitride, zirconium oxide, tantalum dioxide, hafnium oxide or hafnium silicate. According to an embodiment of the present invention, the substrate comprises glass or a flexible insulating polymer, wherein the flexible insulating polymer may include a material selected from the group consisting of polyimide (PI), polystyrene (PS), polymethylmethacrylate (PMMA), polyethylene terephthalate (PET), polycarbonate (PC) and polyvinylchloride (PVC).

The method for fabricating a biosensor for detecting at least a single cell of this invention is described as follows. A substrate having an insulating surface is provided. A conductive layer with a first pattern and a second pattern separated from each other is formed on the substrate. The first pattern has a plurality of first finger configurations and the second pattern has a plurality of second finger configurations, wherein the first and the second finger configurations are interdigitated. A plurality of capture molecules are then immobilized on the conductive layer, such that the cell which is bound specifically to the capture molecules on two adjacent first and second finger configurations is detected.

According to an embodiment of the present invention, forming the conductive layer comprises a patterning step that utilizes lithography and etching or, in the alternative, a lift-off process.

According to an embodiment of the present invention, immobilizing the capture molecules on the conductive layer comprises forming a self-assembled monolayer, including 11-mercaptoundecanoic acid, on the conductive layer, and then forming a layer of the capture molecules on the self-assembled monolayer.

In summary, the biosensor structure for cell detection can be carried out by immobilizing the targeted cell specifically on two adjacent finger configurations of the conductive layer via the capture molecules, i.e. antibodies. Thus, the electrical conductivity of the targeted cell across two adjacent finger configurations can be measured, such that it is possible for the application in real-time, specific, and quantitative cell detection down to a single cell. In addition, the biosensor structure can be applied to various-types of the cells with the modification of the patterned conductive layer and capture molecules. The fabrication of the biosensor structure can be incorporated with the semiconductor process so as to fulfill mass production and cost reduction.

In order to make the aforementioned and other features and advantages of the present invention more comprehensible, preferred embodiments accompanied with figures are described in detail below.

### BRIEF DESCRIPTION OF THE DRAWINGS

The file of this patent contains at least one drawing executed in color Copies of this patent with color drawing(s) will be provided by the Patent and Trademark Office upon request and payment of the necessary fee.”

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic top view of a biosensor structure according to an embodiment of this invention.

FIG. 2 is schematic cross-sectional view of the biosensor structure in FIG. 1 along line I-I.

FIGS. 3A-3C depict, in a cross-sectional view, a method for fabricating a biosensor structure according to an embodiment of this invention.

FIGS. 4A-4C depict, in a cross-sectional view, a method for fabricating a biosensor structure according to another embodiment of this invention.

FIGS. 5A-5B illustrate, in a schematic top view, a process of detecting cells using a biosensor structure according to this invention.

FIGS. 6A and 6B respectively show an OM image and an AFM image of E. coli (JM 109) immobilized on two adjacent finger configurations obtained in an example of this invention.

FIG. 7A illustrates the increased current after E. coli cells immobilization (I_{Antibody+E. coli}−I_{Antibody-only}) measured at 0.5V using a biosensor structure according to this invention versus the number of immobilized E. coli cells.

FIG. 7B illustrates the current of the current measured at 0.5 V using a biosensor structure according to this invention before antibody-modification (Iₙₒₙₑ), after -antibody-modification (I_{Antibody-only}), and after four E. coli cells immobilizes (I_{Antibody+E. coli}).

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

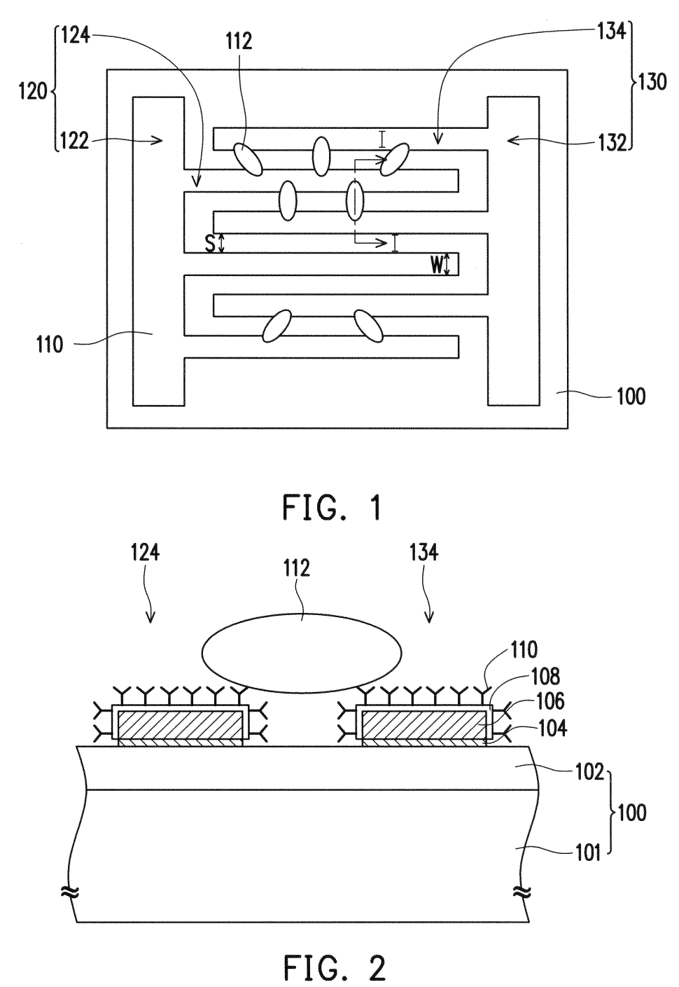

Reference is made to FIG. 1, which is simplified a top view of a biosensor structure according to an embodiment of this invention. The biosensor structure for detecting at least a single cell 112 includes a substrate 100 and two electrodes 120 and 130. The substrate 100 has an insulating surface, and the electrodes 120 and 130 may include a conductor. The electrodes 120 and 130 are disposed on the insulating surface of the substrate 100 and separated from each other. Additionally, each of the electrodes 120 and 130 has a pattern including a body 122 or 132 and a plurality of finger configurations 124 or 134. The bodies 122 and 132 are disposed opposite to each other with the finger configurations 124 and 134 interdigitated between them. Each of the finger configurations 124 and each of the finger configurations 134 are disposed in parallel alternately without contacting one another. Therefore, the electrodes 120 and 130 are designed to detect the electrical conductivity of the cell 112 across two adjacent finger configurations 124 and 134 due to the lack of the structural connection therebetween.

As shown in FIG. 1, the number of finger configurations 124 or 134, the line-width W of each finger configuration 124 or 134, and the spacing S between two adjacent finger configurations 124 and 134 can be varied, depending on the size of the cell 112 to be determined. It is noted that the line-width W needs to be smaller than the size of targeted cell 112 to avoid its immobilization on only one finger configuration leading to no current contribution. In an embodiment, the cell 112- to be detected is E. coli, a bacterium that naturally occurs in the intestinal tracts of humans and warm-blooded animals, having the size within the range of 2-6 μm in length typically.

While a strain of E. coli (JM109) is taken as an example, the line-width W of each finger configuration 124 or 134 ranges between 2 and 6 μm and the spacing S between two adjacent finger configurations 124 and 134 ranges between 1 and 5 μm. In an example, the interdigitated finger configurations 124 and 134 constitute a sensing array with the line-width W and the spacing S designed to be 4 μm and 2 μm-4 μm, respectively.

FIG. 2 is schematic cross-sectional view of the biosensor structure in FIG. 1 along line I-I. Referring to FIGS. 1 and 2, the substrate 100 which has the insulating surface may include a silicon layer 101 and a dielectric layer 102 disposed over the silicon layer 101. The dielectric layer 102 can be silicon dioxide, silicon nitride, or any other suitable dielectric material having a high dielectric constant, e.g. zirconium oxide, tantalum dioxide, hafnium oxide and hafnium silicate. The dielectric layer 102 has a thickness within the range of 5 nm to 500 nm. In another embodiment, the substrate 100 may be an insulator without the dielectric layer disposed thereover, which includes a material selected from the group consisting of glass, polyimide (PI), polystyrene (PS), polymethylmethacrylate (PMMA), polyethylene terephthalate (PET), polycarbonate (PC), polyvinylchloride (PVC) and any other flexible insulating polymers.

The electrodes 120 and 130 may include a conductive layer 106 and a plurality of capture molecules 110. The conductive layer 106 is disposed on the substrate 100, and the capture molecules 110 are immobilized on the surface of the conductive layer 106. The material of the conductive layer 106 can be gold (Au), aluminium (Al), platinum (Pt), or any other suitable metals or conductors. The capture molecules 110 are, for example, antibodies or antibody fragments which can bind to a specific antigen presented by the targeted cell 112. The capture molecules 110 are, for example, covalently linked onto the surface of the conductive layer 106 via a self-assembled monolayer 108 using amine coupling chemistry to promote the immobilization of the capture molecules 110. The self-assembled monolayer 108, for example, includes 11-mercaptoundecanoic acid for modifying the surface of the conductive layer 106 with thiols. Accordingly, the cell 112 binds specifically to the capture molecules 110 immobilized firmly on the finger configurations 124 and 134 through antibody-antigen interactions, and therefore can be detected utilizing the electrical conductivity thereof. It is noted that the materials of the conductive layer 106 and of the self-assembled monolayer 108 is not particularly limited to those illustrated above, and alterations thereof are allowed in the present invention as long as the self-assembled monolayer 108 enables the capture molecules 110 to be immobilized stably on the conductive layer 106.

In an embodiment, an adhesion layer 104 can be deployed between the substrate 100 and the conductive layer 106 to further enhance adhesion between the dielectric layer 102 and the conductive layer 106. The material used as the adhesion layer 104 is, for example, a refractory metal, or a nitride or an alloy thereof, such as titanium (Ti), titanium nitride, tungsten (W), tungsten nitride, titanium-tungsten alloy, tantalum (Ta), tantalum nitride, nickel (Ni), nickel-vanadium alloy, chromium (Cr), etc.

Methods for fabricating the foregoing biosensor structure according to two embodiments of this invention are then described. The following fabricating methods merely demonstrate the procedures for constructing biosensor structure, as shown in FIGS. 1 and 2, in detail, which enable one of ordinary skill in the art to make the biosensor structure claimed in this invention by means of the semiconductor process, but does not limit the scope of this invention.

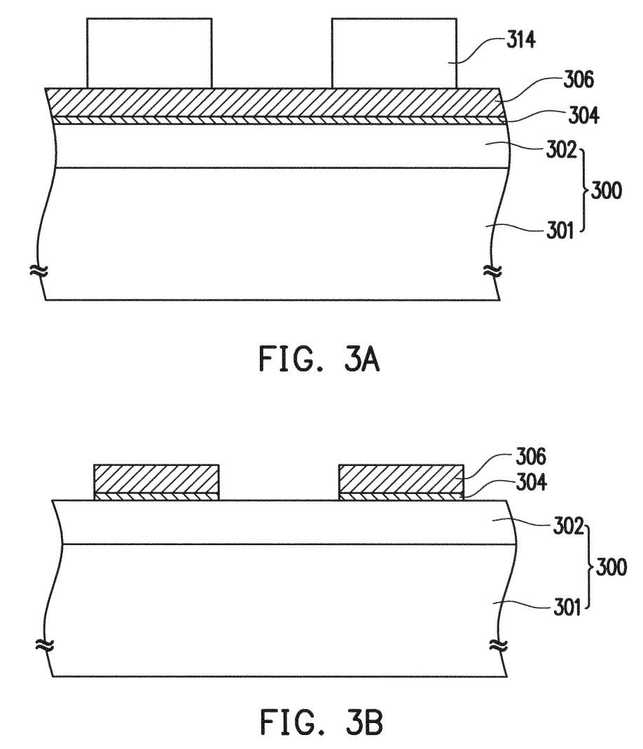

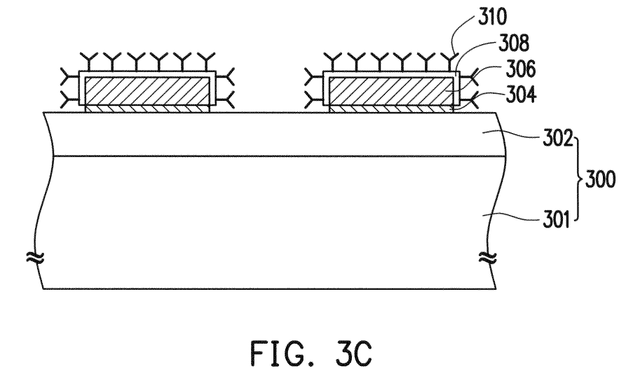

FIGS. 3A-3C depict, in a cross-sectional view, a method for fabricating a biosensor structure according to an embodiment of this invention. Referring to FIG. 3A, a substrate 300 is provided, which has an insulating surface. The substrate 300 may include a silicon layer 301 and a dielectric layer 302, wherein the dielectric layer 302 with a thickness within the range of 5 nm to 500 nm is formed over the silicon layer 301. The material of the dielectric layer 302 can be silicon dioxide, silicon nitride, or any other suitable dielectric material having a high dielectric constant, e.g. zirconium oxide, tantalum dioxide, hafnium oxide and hafnium silicate. The forming method thereof can be chemical vapour deposition (CVD), plasma enhanced CVD (PECVD) or thermal oxidation in case of silicon oxide, and can be CVD, atomic layer deposition (ALD), evaporation or sputtering in case of zirconium oxide, tantalum dioxide, hafnium oxide and hafnium silicate. In another embodiment, the substrate 300 may be an insulator, which includes a material selected from the group consisting of glass, polyimide (PI), polystyrene (PS), polymethylmethacrylate (PMMA), polyethylene terephthalate (PET), polycarbonate (PC), polyvinylchloride (PVC) and any other flexible insulating polymers.

Then, an adhesion layer 304, a conductive layer 306 and a mask layer 314 are formed sequentially on the dielectric layer 302, wherein the adhesion layer 304 can be formed optionally to enhance adhesion between the dielectric layer 302 and the conductive layer 306. The adhesion layer 304 may include a refractory metal, such as titanium (Ti), formed by a deposition step. The conductive layer 306 is formed from gold (Au), aluminium (Al), platinum (Pt), or any other suitable metals or conductors, possibly by sputtering, electroless plating, etc. The mask layer 314 is, for example, a patterned photoresist (PR) layer formed by lithography process, such that partial surface of the conductive layer 306 is exposed in the opening of the mask layer 314. The mask layer 314 may have the pattern corresponding to the patterned conductive layer to be formed in the subsequent process, that is, the pattern with plural finger configurations interdigitated as shown in FIG. 1. Referring to FIG. 3B, the exposed conductive layer 306 and adhesion layer 304 are removed by etching process using the mask layer 314 as a mask. Therefore, the patterned conductive layer 306 may include the finger configurations interdigitated. It is noted that the line-width and spacing of the patterned conductive layer 306 can be designed according to the size of the targeted cell, so as to meet the requirement for different cell detection. The mask layer 314 is removed thereafter.

Referring to FIG. 3C, capture molecules 310 are immobilized on the surface of the conductive layer 306. Prior to the immobilization of the capture molecules 310, the conductive layer 306 may be cleaned by organic solvents (acetone and ethanol) and piranha solution (1:3 H₂O₂-concentrated H₂SO₄) for 1 minute, rinsed with ethanol, and then dried with a flow of N₂ to obtain a clean surface thereof. The immobilization of the capture molecules 310 may be carried out by forming a self-assembled monolayer 308 on the surface of the conductive layer 306 and forming a layer of the capture molecules 310 on the self-assembled monolayer 308 by means of amine coupling chemistry.

More specifically, the self-assembled monolayer 308 includes, for example, 11-mercaptoundecanoic acid for modifying the surface of the conductive layer 306 with thiols. The capture molecules 310 can be antibodies or antibody fragments which bind to a specific antigen presented by the targeted cell, so as to achieve the specific cell detection. The method for forming the self-assembled monolayer 308 can be immersing the conductive layer 306 in an ethanol solution of 1 mM 11-mercaptoundecanoic acid for 12 hours, and then rinsing the same with ethanol to remove the non-bonded thiols. Thereafter, the thiol-modified conductive layer 306 is, for example, treated with 0.4 mM N-ethyl-N′-(3-dimethylaminopropyl)carbodiimide (EDC)-0.1 mM N-hydroxysuccinimide (NHS) for an hour to convert the terminal carboxylic group of 11-mercaptoundecanoic acid to an NHS active ester. After rinsing the thiol-modified conductive layer 306 with deionized (DI)-water and drying it in a flow of N₂, 5 mg/ml of anti-rabbit IgG is dropped onto the surface at 37° C. for an hour to covalently link the capture molecules 310 on the conductive layer 306 via the self-assembled monolayer 308. In addition, the antibody-modified conductive layer 306 is treated with 0.1% bovine serum albumin (BSA) for 35 minutes to block the untreated and non-specific sites after the excess antibodies are removed with phosphate buffered saline (PBS). After rinsing with PBS and DI-water, the biosensor structure is dried with N₂, and therefore is ready.

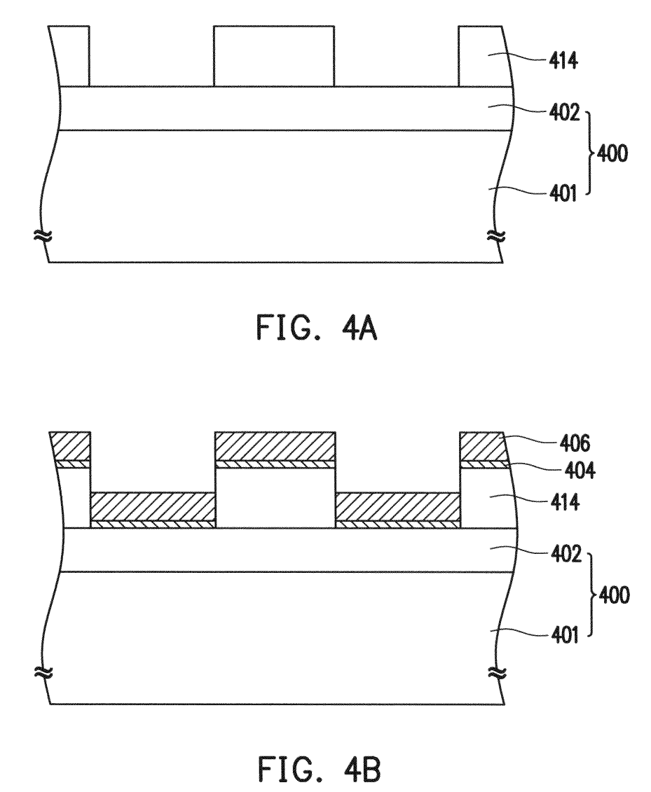

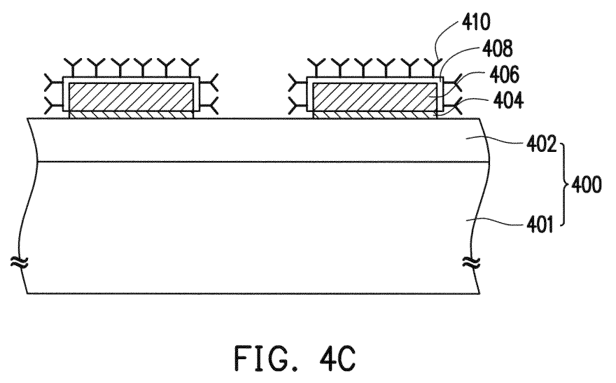

FIGS. 4A-4C depict, in a cross-sectional view, a method for fabricating a biosensor structure according to another embodiment of this invention. The constructing elements of the biosensor structure are roughly identical to those shown in FIGS. 3A-3C, while the difference is in the patterning step of the conductive layer, and hence, detailed descriptions of the same or like elements are omitted hereinafter. Referring to FIG. 4A, a substrate 400 is provided, which may include a silicon layer 401 and a dielectric layer 402 with a thickness within the range of 5 nm to 500 nm over the silicon layer 401. Likewise, in another embodiment, the substrate 400 may be an insulator, which includes a material selected from the group consisting of glass, polyimide (PI), polystyrene (PS), polymethylmethacrylate (PMMA), polyethylene terephthalate (PET), polycarbonate (PC), polyvinylchloride (PVC) and any other flexible insulating polymers. Then, a mask layer 414 is formed on the substrate 400. The mask layer 414 is important as determining the patterned conductive layer formed later, and possibly has the pattern with plural finger configurations interdigitated as shown in FIG. 1. One may use, for example, a patterned photoresist (PR) layer formed by lithography process as the mask layer 414.

Referring to FIG. 4B, an adhesion layer 404 and a conductive layer 406 are formed sequentially on the substrate 400, wherein the adhesion layer 404 can be formed optionally to enhance adhesion between the dielectric layer 402 and the conductive layer 406. Since partial surface of the dielectric layer 402 is covered by the mask layer 414, parts of the conductive layer 406 are deposited on the mask layer 414.

Referring to FIG. 4C, the conductive layer 406 is patterned by removing the mask layer 414 utilizing a lift-off process. Parts of the conductive layer 406 formed on the mask layer 414 are removed simultaneously with the removal of the mask layer 414, so as to accomplish the patterning of the conductive layer 406. The lift-off process may be executed by using a solvent to strip away the mask layer 414, or with the assistance by using ultrasonic activation force. Thereafter, capture molecules 410 are immobilized on the conductive layer 406 via a self-assembled monolayer 408 in a similar manner described in FIG. 3C.

In the field of bacteria detection, a practical example of the method for detecting and quantifying the cells utilizing the biosensor structure according to this invention is provided below. It is to be understood that this specification and the following example are intended to exemplify the real-time, specific and quantitative detection only and thereby enable those of ordinary skill in the art to practice this invention, but are not intended to limit the scope of this invention. It is appreciated by those of ordinary skill in the art that the present invention can be applied to other targeted cells in a manner illustrated in the following example with proper modifications according to known knowledge in the art.

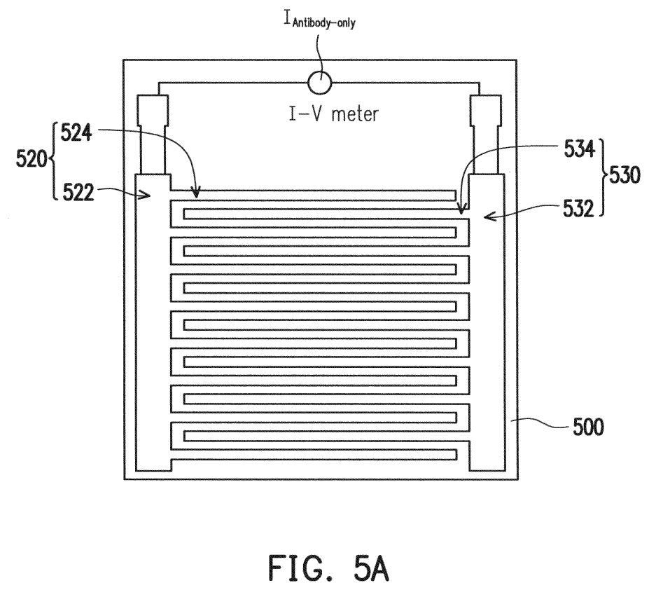

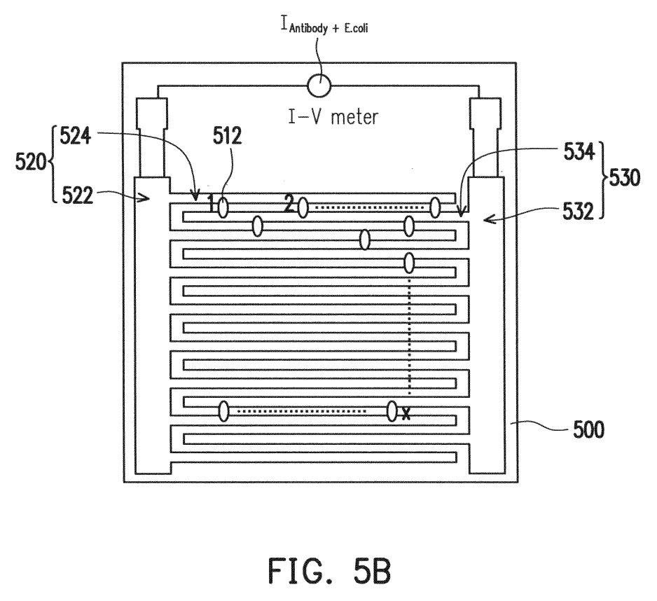

FIGS. 5A-5B illustrate, in a schematic top view, a process of detecting cells using a biosensor structure according to this invention.

Referring to FIG. 5A, the biosensor with the structure described above is provided, wherein the electrodes 520 and 530 with a pattern including the body 522 or 532 and the finger configurations 524 or 534 are disposed on the insulating surface of the substrate 500. Following the antibody-modification on the surface of the electrodes 520 and 530, the electrodes 520 and 530 including 200 finger configurations and 199 spacings in total number form the interdigitated array with a sensing area of 1.2 mm×1.0 mm. Since the detected cell is E. coli (JM 109) with the size within the range of 2-6 μm in length, the line-width of each finger configuration 524 or 534 and the spacing between two adjacent finger configurations 524 and 534 are designed to be 4 μm and 2-4 μm, respectively. To calibrate the current contribution of each E. coli cell by using the interdigitated electrode array composed of the finger configurations 524 and 534, low background current of the antibody-modified electrodes 520 and 530 needs to be ensured before bacteria detection. The current (I_{Antibody-only}) is measured on the electrodes 520 and 530 at a fixed voltage (V=0.5V) after antibody-modification as the background current, which is smaller than 0.7 pA in this case. The I_{Antibody-only} stands for the current measured on the electrodes 520 and 530 after antibody-modification but before E. coli immobilization. The electrical measurement is conducted using HP4155C system.

Referring to FIG. 5B, a sample of 0.5 μl DI-water droplet containing E. coli (JM 109) 512 is positioned right onto the center of the interdigitated array to ensure all E. coli cells can be located inside the array and left for 15 seconds to bind E. coli 512 with the antibodies immobilized thereon. The targeted E. coli 512 is then immobilized on two adjacent finger configurations 524 and 534 attributed to the specific binding between E. coli 512 and the coated antibodies. After the test structure is washed by DI-water for 30 seconds, the sample containing E. coli is baked at 50° C. for 1 minute in air to minimize the background current level resulted from residual moisture or hydration more effectively. Then, the current is measured at 0.5V after E. coli cells immobilizes (I_{Antibody+E. coli}). The I_{Antibody+E. coli} stands for the current measured on the electrodes 520 and 530 after E. coli cells immobilization. The current measured may be varied according to the number of the immobilized E. coli 512 due to the electrical conductivity of E. coli 512 between two adjacent finger configurations 524 and 534.

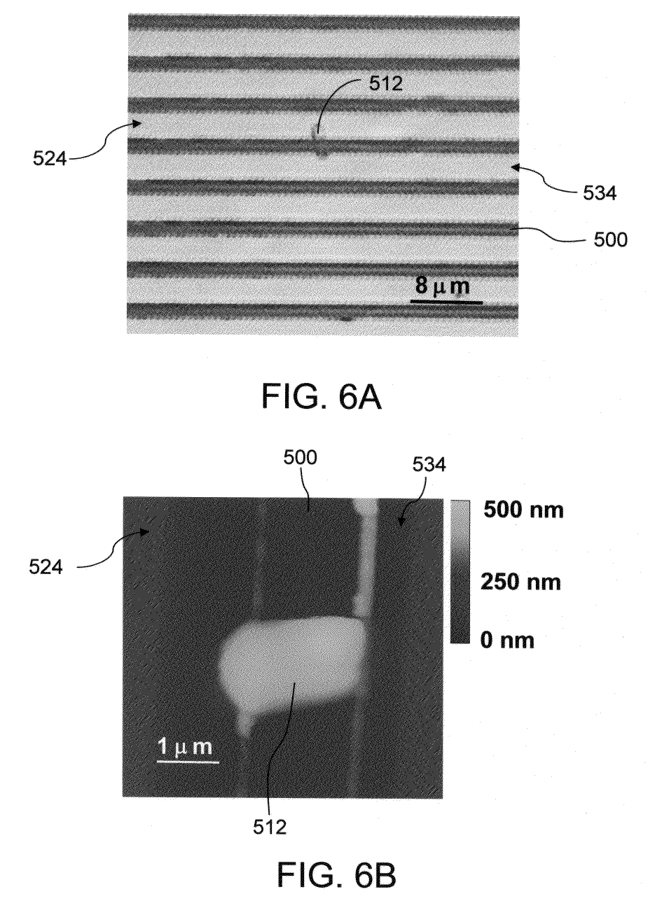

Assuming the current contribution of each E. coli immobilized (Io) is the same, the increased current (ΔIₜ) will be proportional to the total number of E. coli cells (x) immobilized on two adjacent finger configurations 524 and 534, i.e. ΔIₜ=(I_{Antibody+E. coli}−I_{Antibody-only})=xIo, as E. coli cells can be treated as conductors connected in parallel. The number of E. coli immobilized on the electrodes is counted based on the observation under optical microscopy (OM) and confirmed by atomic force microscopy (AFM). FIG. 6A shows the targeted cell 512, i.e. E. coli cell (JM 109), immobilized on two adjacent antibody-modified finger configurations 524 and 534 under OM observation, from which the total number of E. coli cells immobilized can be counted. AFM is also utilized to ensure the immobilization of the targeted cell 512, i.e. E. coli, on two adjacent antibody-modified finger configurations 524 and 534, as shown in FIG. 6B taken by AFM using height-imaging.

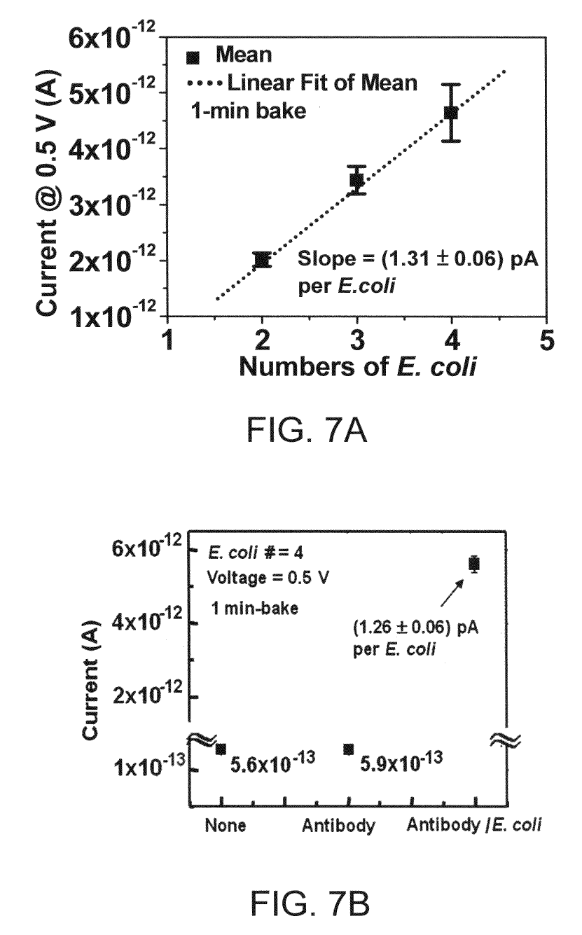

FIG. 7A illustrates the increased current after E. coli cells immobilization (I_{Antibody+E. coli}−I_{Antibody-only}) measured at 0.5V using a biosensor structure according to this invention versus the number of immobilized E. coli cells. To calculate the current contribution of each E. coli (Io) at a fixed voltage (V=0.5V), the current contribution of each E. coli cell (Io) can be obtained from the slope of increased-current (ΔIₜ) measured versus immobilized E. coli number (x) after deducing their background current. From the curve fitting of FIG. 7A, the current contributed by each E. coli cell (Io) is about 1.31±0.06 pA (n=3).

FIG. 7B illustrates the current of the current measured at 0.5 V using a biosensor structure according to this invention before antibody-modification (Iₙₒₙₑ), after antibody-modification (I_{Antibody-only}), and after four E. coli cells immobilizes (I_{Antibody+E. coli})

A simpler way to calculate the current contribution of each immobilized E. coli cell (Io) can be practiced by dividing the increased-current (ΔIₜ) with the number of immobilized E. coli cells (x) directly. Based on above, the current contribution of each E. coli (Io) is calculated to be about 1.26±0.06 pA (n=3), as shown in FIG. 7B, of which the value is consistent with those measured from FIG. 7A. As a comparison, a droplet of 0.5 μl DI-water without E. coli is also positioned onto antibody-modified electrode array followed by 1-min bake at 50° C. as a control, and the results show no current change. This indicates that the electrodes 520 and 530 are indeed electrically connected by the immobilized E. coli, and the current measured in FIG. 7B is mainly contributed by the four E. coli cells immobilized between the finger configurations 524 and 534.

As the current contribution of each E. coli (Io) is obtained from the approach illustrated in FIG. 7A or 7B, a sample containing E. coli with an unknown quantity can be quantified by regressing the increased current (I_{Antibody+E. coli}−I_{Antibody-only}) contributed by the whole E. coli of the sample immobilized onto the interdigitated finger configurations. Therefore, a real-time, specific and quantitative detection of bacteria detection within 3 minutes, which can even determine down to single bacterium, is achieved. It is noted that for the implementation of this approach and extending its application on detecting different types of bacteria or cells, it requires more specific design patterns of the electrodes considering the size and concentration of the targeted cells.

In view of the above, the biosensor structure used for cell detection and the fabrication thereof can be carried out by immobilizing the capture molecules, i.e. antibodies, on the patterned conductive layer. Since the antibody-modified conductive layer disposed on the insulating material has the pattern with interdigitated finger configurations, the targeted cells bound specifically on two adjacent finger configurations via the capture molecules can be detected which may be dominated by the electrical conductivity of the immobilized cells. The current contribution of a single cell can be measured and calibrated by this invention, and hence the biosensor structure is feasible for real-time (<3 min), specific, and quantitative cell detection, i.e. bacterium detection, even down to a single cell.

Furthermore, the interdigitated electrode array used in this prevention is a simple and useful test pattern that can be mass-produced at low cost by incorporating the semiconductor process into the fabrication. The interdigitated electrode array can also be applied to different cell detection, as long as the line-width and spacing of the pattern meet the requirements for the size of the targeted cells so as to effectively immobilize the targeted cell on two adjacent finger configurations.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A biosensor structure for detecting at least a single cell, comprising:
a substrate having an insulating surface;
a conductive layer, disposed on the substrate and having a first pattern and a second pattern, wherein the first pattern having a plurality of first finger configurations and the second pattern having a plurality of second finger configurations are separated from each other, of which the first and the second finger configurations are interdigitated; and
a plurality of capture molecules, immobilized on the conductive layer such that the cell which is bound specifically to the capture molecules on two adjacent first and second finger configurations is detected.

2. The biosensor structure according to claim 1, wherein the capture molecules are antibodies or antibody fragments.

3. The biosensor structure according to claim 1, further comprising a self-assembled monolayer disposed between the conductive layer and the capture molecules.

4. The biosensor structure according to claim 1, wherein the conductive layer comprises gold (Au), aluminium (Al) or platinum (Pt).

5. The biosensor structure according to claim 1, wherein the substrate comprises a silicon layer and a dielectric layer.

6. The biosensor structure according to claim 5, wherein the dielectric layer comprises silicon dioxide, silicon nitride, zirconium oxide, tantalum dioxide, hafnium oxide or hafnium silicate.

7. The biosensor structure according to claim 1, wherein the substrate comprises glass or a flexible insulating polymer.

8. The biosensor structure according to claim 7, wherein the flexible insulating polymer comprises a material selected from the group consisting of polyimide (PI), polystyrene (PS), polymethylmethacrylate (PMMA), polyethylene terephthalate (PET), polycarbonate (PC) and polyvinylchloride (PVC).

9. The biosensor structure according to claim 1, wherein the cell is a bacterium cell.

10. A method for fabricating a biosensor for detecting at least a single cell, comprising:
providing a substrate having an insulating surface;
forming a conductive layer with a first pattern and a second pattern on the substrate, wherein the first pattern having a plurality of first finger configurations and the second pattern having a plurality of second finger configurations are separated from each other, of which the first and the second finger configurations are interdigitated; and
immobilizing a plurality of capture molecules on the conductive layer, such that the cell which is bound specifically to the capture molecules on two adjacent first and second finger configurations is detected.

11. The method according to claim 10, wherein forming the conductive layer comprises a patterning step that utilizes lithography and etching.

12. The method according to claim 10, wherein forming the conductive layer comprises a patterning step that utilizes a lift-off process.

13. The method according to claim 10, wherein the capture molecules are antibodies or antibody fragments.

14. The method according to claim 10, wherein immobilizing the capture molecules on the conductive layer comprises:
forming a self-assembled monolayer on the conductive layer; and
forming a layer of the capture molecules on the self-assembled monolayer.

15. The method according to claim 10, wherein the conductive layer comprises gold (Au), aluminium (Al) or platinum (Pt).

16. The method according to claim 10, wherein the substrate comprises a silicon layer and a dielectric layer.

17. The method according to claim 16, wherein the dielectric layer comprises silicon dioxide, silicon nitride, zirconium oxide, tantalum dioxide, hafnium oxide or hafnium silicate.

18. The method according to claim 10, wherein the substrate comprises glass or a flexible insulating polymer.

19. The method according to claim 18, wherein the flexible insulating polymer comprises a material selected from the group consisting of polyimide (PI), polystyrene (PS), polymethylmethacrylate (PMMA), polyethylene terephthalate (PET), polycarbonate (PC) and polyvinylchloride (PVC).

20. The method according to claim 10, wherein the cell is a bacterium cell.
